SEQUENCE LISTING (15 sequences)

SEQ ID NO: 1 - DNA, 37 bp, synthetic construct
  source 1..37: mol_type = other DNA; note = Primer Gpd-F
ggagatctgatatcactagtattaaacatggcacact

SEQ ID NO: 2 - DNA, 40 bp, synthetic construct
  source 1..40: mol_type = other DNA; note = Primer Gpd-R
atggggctctgttgactcattttgcgtgtgtgtatatgga

SEQ ID NO: 3 - DNA, 20 bp, synthetic construct
  source 1..20: mol_type = other DNA; note = Primer Mmd-F
atgagtcaacagagccccat

SEQ ID NO: 4 - DNA, 25 bp, synthetic construct
  source 1..25: mol_type = other DNA; note = Primer Mmd-R
gggaattcattcagaacggagaagt

SEQ ID NO: 5 - DNA, 1111 bp, synthetic construct
  source 1..1111: mol_type = other DNA; note = Methylmercury demethylase gene Mmd
atgagtcaacagagccccatcaagattcaactgctgaccgttccagactgcccgttggtagccaaggtccgtgacacactcaacaactgcttggcaaaaacacgctcaggcgcaacggtcgaggagcttgtgggagagtatcattctccaacgcttctcatcaacggcttcgacgtcaccggtaagcccgtctcagcacaaggacaacaaagctgccgtctcgatcttcccaatgaggagcagatacttgctgctcttcggggcctgcccgttctaagttgcgaggatgaaacagaggcagcggtgggaaagtctgcttttcacattctgctacgtaccgcagggcgtgtcgcattggaacaggtcagccaagagactggtagaaatacggacgacatcaggaccggtatagaggcgcttcggcgcaggggccatgtcaagatagataaacaaggcttcatcattggggttgctggattgagttgcattcccacagagcatcaactttccatcgaggggaagagactctgggcttggtgtgcctttgacgtgattggcatttttggtgctcttgaggcgtctggattcgcgacatcggcagacccggctactaacgagcgcttggtcgtcaactttgtcaagggtgttcctgatgagacggggcttggggttttcatggcagacatgccaccggggggctctgtctgcgaggactggtgttggagagtgcgattttttcagtccgagtcggcggctgaggcttgggcccgggcgaacggtgtcacgggttctcttatatctgtggcaaacctgatggtatcggcccgcgaagcatggagtaggtacgggttgtcttgaggatagactggcggaattggtatgacctgccatctgagagcgggaagctccatgcaagtcatagttctctcagctctgtaaagatgacttcttgttcgtgttttctatggtgtgttcgtgatagcagagtggcggttcagaagaaataatgattctagctactaggtggacgtttcttaattaaaccggtcgacatgtccaagtacacagttgctctctccgaaatgtccgcattcacttctccgttctgaat

SEQ ID NO: 6 - DNA, 1495 bp, synthetic construct
  source 1..1495: mol_type = other DNA; note = Promoter of 3-phosphoglyceraldehyde dehydrogenase gene Gpd
acatggcacactggtggagacgatttcatggatggcaagactaagcattgggcccagacacgggggttgacacacgggcaggttgccccggtccaggtggaagtttacgattgaaaacttccgaagagcttgtattccctgtgggcaaagttcgggacgacgagaactgttcctggtgttcccgacgactgtccccgggcggacgcggctgcaaaggccagagccgcccgcagccgtgaagctccgggaaagatgggcaaaacgggcgaatggatggccaatacagatgaaccatttcccgccatgcatccgagccgcccacgcgaccagaggggccccggaattaattagattgaggtcatcgtgaaggcattccgctgctgagagtgcgacagagctggcgtcggtacattttggacatgtctgggccgtctgatttttttttccgtctcctctcacacctgactgcgtgagtcagcagcaagtcgtcaccgcgaccggagacacgttttgagtgatttatcatggctattactcgcggtcttgctgccagcaggcatatctacaacacgataaggccagcagggcctaccacgggggtacatggacgccaggctgcgacaaccatggaacagcgggtacccctcatgtccatgttttttttctctctctcaaggtggtgctcatgtgctcgtcggtgtggttgagagagcgtttctattaacgaattgtgtccccgccggatccgtaaaaaagacgcatcggctgggtggagcaaggcgaggcccgtcaaagcatcatcaatggctgagctcattcgattcctcatcaaattacatggcgcagggaagaatcgctggcgaattttgcctttgcggtgcatgcaagtggcggctggctgggatattggatggccacttgcacgaccacggcgaggatggacatgaagcccgcggcgtctggcatggcaaggcaattgaaatatcgtctcgttgaatagcgtgaatagtagtaccgctcgtgtatcagaacccggctccgtctcacggtgcaagtgtgaatgatgcatgcacccgcttggctaggtttgcgtgcaccagttgttggccgctgacgtctgctggtctcaacagtcccgacgccgtggccagccaagacgcagccagttgcgcgcccagcaacacaccagacaccccaagcaccagacccaagctcccaagctccagggacatggactgaccactattttgtcccgccttccctccgtcccctccgactccatcttcttccctcttcctcttcctttttcttcttctcttcccgctctccatacaacttcaccattcttcccaccaggcactttgtccggtatgtatcccgtcctgcattggctgagcagagcttgaccacagctcgacaccagctccgtctctagcttcccagactcccgtgctgacgtccaagctccagctctctccatatacacacacgcaaa

SEQ ID NO: 7 - DNA, 28 bp, synthetic construct
  source 1..28: mol_type = other DNA; note = Primer Tef-EcoRI-F
ccgaattctagcaacagtcagctagacg

SEQ ID NO: 8 - DNA, 28 bp, synthetic construct
  source 1..28: mol_type = other DNA; note = Primer Trpc-XbaI-R
gctctagagcattgcagatgagctgtat

SEQ ID NO: 9 - DNA, 18 bp, synthetic construct
  source 1..18: mol_type = other DNA; note = Primer Tps-F
atggcccagatctccatc

SEQ ID NO: 10 - DNA, 19 bp, synthetic construct
  source 1..19: mol_type = other DNA; note = Primer Tps-R
ttagatggggacctggtcg

SEQ ID NO: 11 - protein, 580 aa, synthetic construct
  source 1..580: note = Protein encoded by Tps
MAQISIGAPLSAEVNGACINTHHHGNLWDDYFIQSLKSPYEAPECHERCEKMIEEVKHLLLSEMRDGNDDLIKRLQMVDIFECLGIDRHFHHEIQAALDYVYRYWNELEGIGVGTRDSLTKDLYATGLGFRALRLHRYNVSSAVLENFKNENGLFFHSSAVQEEEVRCMLTLLRASEISFPGEKVMDEAKAFATEYLNQLLTRVDITEVGENLLREVRYALDFPWYCSVPRWEARSFIEIFGQNNSWLKSTMNKKVLELAKLDFNILQSAHQRELQLLSRWWSQSDIEKQNFYRKRHVEFYFWMVIGTFEPEFSSSRIAFAKIATLMTILDDLYDTHGTLEQLKIFTEAVKRWDLSLQDRLPDYIKITLEFFFNTSNELNAEVAKMQERDMSAYIRKAGWERYIEGYMQESEWMAARHVPTFDDYMKNGKRSSGMCILNLYSLLLMGQLVPDNILEQIHLPSKIHELVELTARLVDDSKDFQAKKDGGEFASGTECYLKEKPECTEEDAMNHLIGLLNLTAMELNWEFVKHDGVALCLKKFVFEVARGLRFIYKYRDGFDYSNEEMKSQITKILIDQVPI

SEQ ID NO: 12 - DNA, 1743 bp, synthetic construct
  source 1..1743: mol_type = other DNA; note = Longifolene synthesis gene Tps
atggcccagatctccatcggcgcccccctctccgccgaggtcaacggcgcctgcatcaacacccaccaccacggcaacctctgggacgactacttcatccagtccctcaagtccccctacgaggcccccgagtgccacgagcgctgcgagaagatgatcgaggaggtcaagcacctcctcctctccgagatgcgcgacggcaacgacgacctcatcaagcgcctccagatggtcgacatcttcgagtgcctcggcatcgaccgccacttccaccacgagatccaggccgccctcgactacgtctaccgctactggaacgagctcgagggcatcggcgtcggcacccgcgactccctcaccaaggacctctacgccaccggcctcggcttccgcgccctccgcctccaccgctacaacgtctcctccgccgtcctcgagaacttcaagaacgagaacggcctcttcttccactcctccgccgtccaggaggaggaggtccgctgcatgctcaccctcctccgcgcctccgagatctccttccccggcgagaaggtcatggacgaggccaaggccttcgccaccgagtacctcaaccagctcctcacccgcgtcgacatcaccgaggtcggcgagaacctcctccgcgaggtccgctacgccctcgacttcccctggtactgctccgtcccccgctgggaggcccgctccttcatcgagatcttcggccagaacaactcctggctcaagtccaccatgaacaagaaggtcctcgagctcgccaagctcgacttcaacatcctccagtccgcccaccagcgcgagctccagctcctctcccgctggtggtcccagtccgacatcgagaagcagaacttctaccgcaagcgccacgtcgagttctacttctggatggtcatcggcaccttcgagcccgagttctcctcctcccgcatcgccttcgccaagatcgccaccctcatgaccatcctcgacgacctctacgacacccacggcaccctcgagcagctcaagatcttcaccgaggccgtcaagcgctgggacctctccctccaggaccgcctccccgactacatcaagatcaccctcgagttcttcttcaacacctccaacgagctcaacgccgaggtcgccaagatgcaggagcgcgacatgtccgcctacatccgcaaggccggctgggagcgctacatcgagggctacatgcaggagtccgagtggatggccgcccgccacgtccccaccttcgacgactacatgaagaacggcaagcgctcctccggcatgtgcatcctcaacctctactccctcctcctcatgggccagctcgtccccgacaacatcctcgagcagatccacctcccctccaagatccacgagctcgtcgagctcaccgcccgcctcgtcgacgactccaaggacttccaggccaagaaggacggcggcgagttcgcctccggcaccgagtgctacctcaaggagaagcccgagtgcaccgaggaggacgccatgaaccacctcatcggcctcctcaacctcaccgccatggagctcaactgggagttcgtcaagcacgacggcgtcgccctctgcctcaagaagttcgtcttcgaggtcgcccgcggcctccgcttcatctacaagtaccgcgacggcttcgactactccaacgaggagatgaagtcccagatcaccaagatcctcatcgaccaggtccccatctaa

SEQ ID NO: 13 - DNA, 20 bp, synthetic construct
  source 1..20: mol_type = other DNA; note = Primer EF1T
atgggtaaggargacaagac

SEQ ID NO: 14 - DNA, 21 bp, synthetic construct
  source 1..21: mol_type = other DNA; note = Primer EF2T
ggargtaccagtsatcatgtt

SEQ ID NO: 15 - DNA, 761 bp, synthetic construct
  source 1..761: mol_type = other DNA; note = Sequencing result of the strain TM1
ttttttttagtggatatgaggggcaagactcacatcaacgtggtcgttatcgtaagtcgcctgcctccatttcgaactttgtagaagctgttgtactgacttgcttgtcgtaggggtatgtttcggagcctacactcttcgccgtcccgagtttgtgataactgactggtcctcacagccacgtcgactccggcaagtctaccaccactggtcacttgatctaccagtgcggtggtatcgacaagcgtaccattgagaagttcgagaaggtaagccaaaccactccgattaatgatctgctattgtttggcgatgaacattattgggtttcccgctgcctgtcggccattacccctcactgtggcacgaaaattttcgcggggccttatcttggactttggtggggcatcataccccgccagctgtcgagggtgtctctgtgtgtctctggctgttgaaaccacaatattgtcgttgctttcagagggaaaaaacatgaaactaatttggatcgctgtataggaagccgctgaactcggcaagggttccttcaagtacgcatgggttcttgacaagctcaaggccgagcgtgagcgtggtatcaccatcgacattgccctctggaagttcgagactcccaagtactatgtcaccgtcattggtatgtcgacttgcgcaaactgaccgcagacttttctcctaaattgaatgctaatgcccctcccacagacgctcccggtcaccgtgactttatcaagaac

(19) United States
(12) Patent Application Publication
(10) Pub. No.: US 2026/0107949 A1
(43) Pub. Date: Apr. 23, 2026

(54) METARHIZIUM PINGSHAENSE STRAIN FOR CONTROLLING SOLENOPSIS INVICTA, AND ENGINEERED STRAIN AND USE THEREOF

(71) Applicant: Zhejiang University, Hangzhou City (CN)

(72) Inventors: Weiguo FANG, Hangzhou City (CN); Meihong NI, Hangzhou City (CN); Yubo ZHANG, Hangzhou City (CN); Mingxing JIANG, Hangzhou City (CN); Xingyuan TANG, Hangzhou City (CN)

(21) Appl. No.: 19/027,077

(22) Filed: Jan. 17, 2025

(30) Foreign Application Priority Data
Oct. 23, 2024 (CN) 202411479420.2

(51) Int. Cl. A01N 63/30; A01N 43/54; A01P 7/04; A01P 19/00; C12N 9/02; C12N 15/80

(52) U.S. Cl. CPC A01N 63/30; A01N 43/54; A01P 7/04; A01P 19/00; C12N 9/0008; C12N 15/80; C12Y 102/01012

(57) ABSTRACT

A Metarhizium pingshaense strain for controlling Solenopsis invicta, and an engineered strain and use thereof are provided, relating to the technical field of controlling Solenopsis invicta. The M. pingshaense strain is a strain isolated from the wild and is highly virulent to the S. invicta. It can be used to control the S. invicta or to construct an engineered strain with improved S. invicta control efficacy. The constructed engineered strain exhibits high virulence to the S. invicta, and enables the ant cadavers infected by the strain to volatilize a large amount of the attractant longifolene of the S. invicta. As a result, the ant cadavers exhibit a luring and killing effect on workers of the S. invicta, significantly limiting the hygienic behavior of the workers.

### CROSS REFERENCE TO RELATED APPLICATION

This patent application claims the benefit and priority of Chinese Patent Application No. 2024114794202 filed with the China National Intellectual Property Administration on Oct. 23, 2024, the disclosure of which is incorporated by reference herein in its entirety as part of the present application.

### REFERENCE TO SEQUENCE LISTING

A computer readable XML file entitled “GWP20240806282_seqlist”, that was created on Nov. 26, 2024, with a file size of about 22,484 bytes, contains the sequence listing for this application, has been filed with this application, and is hereby incorporated by reference in its entirety.

### TECHNICAL FIELD

The present disclosure relates to the technical field of Solenopsis invicta prevention and control, in particular to a Metarhizium pingshaense strain for controlling S. invicta, and an engineered strain and use thereof.

### BACKGROUND

The red imported fire ant (Solenopsis invicta), a significant invasive pest, exhibits varied diets, strong aggressiveness, and high reproductive capacity. It poses serious threats to agricultural and forestry production, human and animal health, public safety, and ecological balance. In recent years, the spread of this species has accelerated in many countries around the world, with notable new outbreaks emerging. At present, the control of Solenopsis invicta (S. invicta) relies on the large-scale application of contact chemical pesticides, which can seriously threaten human health and biodiversity while posing multiple risks such as pesticide resistance and environmental pollution. Therefore, it is crucial to research and develop environmentally friendly biological control methods for the sustainable management of S. invicta, as this will play a crucial role in future effective control strategies for S. invicta.

Researchers both domestically and internationally have been conducting studies on the biological control of S. invicta for many years. These studies primarily focus on screening for highly effective strains of insecticidal fungi, such as Metarhizium and Beauveria, as well as developing supporting application techniques. Currently, several strains that are highly virulent to the S. invicta have been identified, but the control effectiveness remains unsatisfactory.

### SUMMARY

In order to solve the above problems, an objective of the present disclosure is to provide a Metarhizium pingshaense (M. pingshaense) strain for controlling S. invicta, and an engineered strain and use thereof. The M. pingshaense strain is a strain isolated from the wild and exhibiting high virulence towards S. invicta, and can be used to control the S. invicta or to construct an engineered strain with improved S. invicta control efficacy.

To achieve the above objective, the present disclosure provides the following technical solutions.

The present disclosure provides a M. pingshaense strain with a deposit number of CGMCC NO. 41511.

The present disclosure further provides use of the M. pingshaense strain in controlling S. invicta or constructing an engineered strain with improved S. invicta control efficacy.

The present disclosure further provides an engineered strain for controlling S. invicta, including a starting strain and a longifolene synthesis gene Tps introduced into the starting strain; where the starting strain includes the M. pingshaense strain; and the longifolene synthesis gene Tps encodes a protein having the amino acid sequence of SEQ ID NO: 11.

In some embodiments, the engineered strain further includes a selection marker gene introduced into the starting strain; the selection marker gene is a methylmercury demethylase gene Mmd of the starting strain, and the methylmercury demethylase gene Mmd has the nucleotide sequence of SEQ ID NO: 5.

In some embodiments, the methylmercury demethylase gene Mmd is driven by a promoter of a 3-phosphoglyceraldehyde dehydrogenase gene Gpd of the starting strain, and the promoter has the nucleotide sequence of SEQ ID NO: 6.

The present disclosure further provides a microbial inoculant for controlling S. invicta, including spore powder, a solid phase carrier, an emulsifier, and a dispersant; where the spore powder includes spore powder of the M. pingshaense strain and/or spore powder of the engineered strain.

In some embodiments, the solid phase carrier includes talc; the emulsifier includes sodium dodecyl sulfate (SDS); and the dispersant includes sodium salt of polynaphthalene sulphonic acid.

The present disclosure further provides use of the engineered strain or the microbial inoculant in controlling S. invicta.

The present disclosure further provides a method for controlling S. invicta, including the following steps: applying a bait for luring and killing the S. invicta to a surface of an S. invicta nest, and then adding a spore suspension to the S. invicta nest after the bait is carried by an ant colony; where a spore in the spore suspension is one or more selected from the group consisting of a spore of the M. pingshaense strain, a spore of the engineered strain, and spore powder in the microbial inoculant.

In some embodiments, the bait for luring and killing the S. invicta includes hydramethylnon.

### Beneficial Effects

The present disclosure provides a M. pingshaense strain with a deposit number of CGMCC NO. 41511. The M. pingshaense strain is a strain isolated from the wild and highly virulent to the S. invicta, and may be used to control the S. invicta or to construct an engineered strain with improved S. invicta control efficacy.

Further, the constructed engineered strain is highly virulent to the S. invicta. The engineered strain enables the ant cadavers resulting from the infection to volatilize a large amount of the attractant longifolene by expressing the longifolene synthesis gene Tps. As a result, the ant cadavers exhibit a luring and killing effect on workers of the S. invicta, significantly limiting the hygienic behavior of the workers. When the engineered strain is used in combination with a small amount of a chemical insecticide bait, the wettable powder with spores of the engineered strain as an active ingredient exerts an efficacy of 79% (58%) within 3 d, 93% (90%) within 7 d, and 98% (98%) within 28 d against multiple-queen nests (single-queen nests) of the S. invicta. Additionally, this method is characterized by its safety and high efficiency.

### BRIEF DESCRIPTION OF THE DRAWINGS

To illustrate the embodiments of the present disclosure or the technical solutions in the prior art more clearly, the accompanying drawings required in the examples will be briefly introduced below.

FIG. 1 shows the results of phylogenetic analysis based on the nucleotide sequences of the TEF gene of strain TM1 (bold) and other taxonomically classified Metarhizium species;

FIGS. 2A-2B show the results of virulence analysis of M. pingshaense on workers of S. invicta; where FIG. 2A shows the median lethal time LT₅₀, with different letters indicating significant differences, namely P<0.05; FIG. 2B illustrates the cumulative mortality rate of workers of S. invicta at different days after inoculation;

FIG. 3 shows the percentage of preference on S. invicta for longifolene; where * represents P<0.01, ** represents P<0.001;

FIG. 4 shows a map of the plasmid pPK2-Mmd-GFP;

FIG. 5 shows a map of the plasmid pPK2-Tps-Mmd-GFP;

FIG. 6 shows the PCR verification results of the genome of the engineered strain Mmd-Tps with Tps insertion;

FIGS. 7A-7E show the analysis results of attractiveness of the cadavers obtained by infecting the workers of S. invicta with the engineered strain Mmd-Tps; where FIG. 7A represents the LT so of the engineered strain Mmd-Tps to the workers of S. invicta; FIG. 7B shows the cadaver formed from Mmd-Tps infection; FIG. 7C shows the amount of longifolene volatilized from the ant cadavers; FIG. 7D illustrates the response index of the workers to cadavers resulting from infection of the engineered strain Mmd-Tps, as detected by a two-way selection method; FIG. 7E shows the results of preference analysis of healthy workers of S. invicta to Mmd-Tps induced cadavers; different letters indicate significant differences, namely P<0.05; FIG. 7B to FIG. 7E are the infection results at a spore concentration of 10⁷ spores/mL;

FIGS. 8A-8B show the results of hygienic behavior analysis of healthy workers in the ant colony towards the cadavers induced-by M. pingshaense strain and its engineered strain Mmd-Tps; where FIG. 8A presents the statistical result of the time when the two types of cadavers began to be abandoned; FIG. 8B shows the proportion of individuals re-inoculated with spores from the body surface of the cadavers during the cadaver removal process; and

FIGS. 9A-9C show the results of combined use of the engineered strain Mmd-Tps and hydramethylnon bait against S. invicta; where FIG. 9A illustrates the control effect on a single-queen nest; FIG. 9B shows the control effect on a multi-queen nest; FIG. 9C shows the relocation rate of the ant nest after treatment; different letters indicate that the effects of different control methods within the same social type are significantly different, namely P<0.05.

### DEPOSIT OF BIOLOGICAL MATERIAL

The M. pingshaense strain was deposited at the China General Microbiological Culture Collection Center (CGMCC), Institute of Microbiology, Chinese Academy of Sciences, Yard 3, NO. 1, West Beichen Road, Chaoyang District, Beijing, China on Sep. 18, 2024 under the deposit number of CGMCC NO. 41511.

### DETAILED DESCRIPTION OF THE EMBODIMENTS

The present disclosure provides a M. pingshaense strain with a deposit number of CGMCC NO. 41511. In the present disclosure, the M. pingshaense strain has an LT₅₀ of only 3.4 d at a spore concentration of 10⁷ spores/mL, and its lethality rate after inoculation for 14 d is 95%. The strain kills S. invicta better than the reported insecticidal fungal strains retrieved.

Based on the above advantages, the present disclosure further provides use of the M. pingshaense strain in controlling S. invicta or constructing an engineered strain with improved S. invicta control efficacy.

The present disclosure further provides an engineered strain for controlling S. invicta, including a starting strain and a longifolene synthesis gene Tps introduced into the starting strain; where the starting strain includes the M. pingshaense strain; and the longifolene synthesis gene Tps preferably encodes a protein having the amino acid sequence of SEQ ID NO: 11:

MAQISIGAPLSAEVNGACINTHHHGNLWDDYFIQSLKSPYEAPECHERCEKMIEE
VKHLLLSEMRDGNDDLIKRLQMVDIFECLGIDRHFHHEIQAALDYVYRYWNELEGIGVG
TRDSLTKDLYATGLGFRALRLHRYNVSSAVLENFKNENGLFFHSSAVQEEEVRCMLTLLR
ASEISFPGEKVMDEAKAFATEYLNQLLTRVDITEVGENLLREVRYALDFPWYCSVPRWEA
RSFIEIFGQNNSWLKSTMNKKVLELAKLDFNILQSAHQRELQLLSRWWSQSDIEKQNFY
RKRHVEFYFWMVIGTFEPEFSSSRIAFAKIATLMTILDDLYDTHGTLEQLKIFTEAVKRWD
LSLQDRLPDYIKITLEFFFNTSNELNAEVAKMQERDMSAYIRKAGWERYIEGYMQESEW
MAARHVPTFDDYMKNGKRSSGMCILNLYSLLLMGQLVPDNILEQIHLPSKIHELVELTAR
LVDDSKDFQAKKDGGEFASGTECYLKEKPECTEEDAMNHLIGLLNLTAMELNWEFVKH
DGVALCLKKFVFEVARGLRFIYKYRDGFDYSNEEMKSQITKILIDQVPI.

As an embodiment, the longifolene synthesis gene Tps has the nucleotide sequence of SEQ ID NO: 12:

5′-atggcccagatctccatcggcgcccccctctccgccgaggtcaacggcgcctgcatcaacacccaccaccacggcaacctctgggacga
ctacttcatccagtccctcaagtccccctacgaggcccccgagtgccacgagcgctgcgagaagatgatcgaggaggtcaagcacctcct
cctctccgagatgcgcgacggcaacgacgacctcatcaagcgcctccagatggtcgacatcttcgagtgcctcggcatcgaccgccacttc
caccacgagatccaggccgccctcgactacgtctaccgctactggaacgagctcgagggcatcggcgtcggcacccgcgactccctcac
caaggacctctacgccaccggcctcggcttccgcgccctccgcctccaccgctacaacgtctcctccgccgtcctcgagaacttcaagaac
gagaacggcctcttcttccactcctccgccgtccaggaggaggaggtccgctgcatgctcaccctcctccgcgcctccgagatctccttccc
cggcgagaaggtcatggacgaggccaaggccttcgccaccgagtacctcaaccagctcctcacccgcgtcgacatcaccgaggtcggc
gagaacctcctccgcgaggtccgctacgccctcgacttcccctggtactgctccgtcccccgctgggaggcccgctccttcatcgagatctt
cggccagaacaactcctggctcaagtccaccatgaacaagaaggtcctcgagctcgccaagctcgacttcaacatcctccagtccgccca
ccagcgcgagctccagctcctctcccgctggtggtcccagtccgacatcgagaagcagaacttctaccgcaagcgccacgtcgagttctac
ttctggatggtcatcggcaccttcgagcccgagttctcctcctcccgcatcgccttcgccaagatcgccaccctcatgaccatcctcgacgac
ctctacgacacccacggcaccctcgagcagctcaagatcttcaccgaggccgtcaagcgctgggacctctccctccaggaccgcctcccc
gactacatcaagatcaccctcgagttcttcttcaacacctccaacgagctcaacgccgaggtcgccaagatgcaggagcgcgacatgtccg
cctacatccgcaaggccggctgggagcgctacatcgagggctacatgcaggagtccgagtggatggccgcccgccacgtccccaccttc
gacgactacatgaagaacggcaagcgctcctccggcatgtgcatcctcaacctctactccctcctcctcatgggccagctcgtccccgaca
acatcctcgagcagatccacctcccctccaagatccacgagctcgtcgagctcaccgcccgcctcgtcgacgactccaaggacttccagg
ccaagaaggacggcggcgagttcgcctccggcaccgagtgctacctcaaggagaagcccgagtgcaccgaggaggacgccatgaacc
acctcatcggcctcctcaacctcaccgccatggagctcaactgggagttcgtcaagcacgacggcgtcgccctctgcctcaagaagttcgt
cttcgaggtcgcccgcggcctccgcttcatctacaagtaccgcgacggcttcgactactccaacgaggagatgaagtcccagatcaccaag
atcctcatcgaccaggtccccatctaa-3′.

The hygienic and nest relocation behaviors of S. invicta result in poor control effect of fungal insecticides. It is found that longifolene has an attractant effect on workers of S. invicta, and the effect is significant when the concentration is 10-3 g/mL, 10-+g/mL, and 10-5 g/mL. The engineered strain constructed using this characteristic and the high virulence of M. pingshaense can effectively kill S. invicta; the engineered strain kills the workers of S. invicta and forms cadavers, which can volatilize the S. invicta attractant longifolene, killing healthy workers and significantly slowing down the hygienic behavior of workers. The engineered strain has a high control effect on both single-queen nest and multiple-queen nest of S. invicta, with a control effect of 90% after 1 week of treatment and 98% after 4 weeks of treatment.

As an embodiment, the selection marker gene of the engineered strain is a methylmercury demethylase gene Mmd of the starting strain, and the methylmercury demethylase gene Mmd has the nucleotide sequence of SEQ ID NO: 5; the methylmercury demethylase gene Mmd is driven by a promoter of a 3-phosphoglyceraldehyde dehydrogenase gene Gpd of the starting strain, and the promoter has the nucleotide sequence of SEQ ID NO: 6. When the insecticidal fungi is genetically transformed to create engineered strains, herbicide resistance genes are generally used as screening marker genes, but these exogenous marker genes are prone to cause concerns about the safety of engineered strains. However, the screening marker gene in the present disclosure is the methylmercury demethylase gene Mmd of the M. pingshaense strain itself, and its expression is also driven by the promoter of the 3-phosphoglyceraldehyde dehydrogenase gene Gpd of the strain itself. That is, the engineered strain does not contain exogenous selection marker genes, so there is no risk of horizontal transfer of exogenous resistance genes, thus ensuring the safety of the engineered strain.

The present disclosure further provides a microbial inoculant for controlling S. invicta, including spore powder, a solid phase carrier, an emulsifier, and a dispersant; where the spore powder includes spore powder of the M. pingshaense strain and/or spore powder of the engineered strain.

As an embodiment, the solid phase carrier may be talc; the emulsifier may be SDS; the dispersant may be sodium salt of polynaphthalene sulphonic acid; the spore powder is a pure spore powder obtained by sieving with a 50-mesh standard sieve (aperture 0.355 mm).

As an embodiment, the microbial inoculant includes the following components in percentage by mass: 35% of the fungal spore powder, 57% of the talc, 3% of the SDS, and 5% of the sodium salt of polynaphthalene sulphonic acid. The microbial inoculant can be mixed with water on site to prepare the spore suspension when controlling S. invicta.

In the present disclosure, the solid phase carrier, emulsifier, and dispersant in the microbial inoculant can be mixed with the spore powder to form wettable powder, such that the spore powder can be better dissolved in water and is easy to use. The solid phase carrier mainly plays two roles. One effect is to act as a tiny container for fungal spores, such that the spores can adhere to the carrier. Meanwhile, the carrier acts as a solid diluent to dilute the spore powder to avoid the spore powder concentration being too high and agglomerating during use. The second effect is to protect and restore the activity of the spores, avoid the spores from losing their vitality after long-term storage, and extend the storage time of the spores. The emulsifier can help the hydrophobic spores to suspend in water, such that the spores can be diluted and diffused, and wetted faster in water. The dispersant can increase the viscosity of the solution, thereby increasing the resistance to the sedimentation of spores, delaying their sedimentation, and improving the suspension stability of the spore suspension.

Based on the above advantages, the present disclosure further provides use of the engineered strain or the microbial inoculant in controlling S. invicta.

The present disclosure further provides a method for controlling S. invicta, including the following steps: applying a bait for luring and killing the S. invicta to a surface of an S. invicta nest, and then adding a spore suspension to the S. invicta nest after the bait is carried by an ant colony; where a spore in the spore suspension is one or more selected from the group consisting of a spore of the M. pingshaense strain, a spore of the engineered strain, and spore powder in the microbial inoculant.

As an embodiment, the bait for luring and killing the S. invicta can be hydramethylnon; the bait for luring and killing the S. invicta is added at 20 g/each ant nest; the ant nest can be a single-queen nest and/or a multiple-queen nest.

In the present disclosure, the method adopts a small amount of chemical bait that kills S. invicta to treat the ant nest, such that the activity of the ant colony decreases, and then a spore suspension prepared from the wettable powder of the engineered strain (or wild-type strain) of M. pingshaense strain is poured into the ant nest. The strain kills S. invicta to form cadavers, which can further lure and kill other individuals in the S. invicta nest, causing the fungus to spread in the ant nest and eventually kill the entire ant nest. The method has a desirable control effect on the S. invicta and greatly reduces the dosage of chemical pesticides, showing a great application prospect.

In order to further illustrate the present disclosure, the M. pingshaense strain for controlling S. invicta, and the engineered strain and the use thereof provided by the present disclosure are described in detail below with reference to the accompanying drawings and examples, but the accompanying drawings and the examples should not be construed as limiting the protection scope of the present disclosure.

### Example 1 Screening of Metarhizium Strain Highly Virulent to S. invicta

A Metarhizium strain was isolated from the grassland soil of Tianmu Mountain in Hangzhou, China and named TM1. The strain was amplified by PCR based on the 5′ end of transcript elongation factor 1-α (5TEF) with primers EF1T and EF2T, which had specific sequenced as follows:

EFIT:
SEQ ID NO: 13
5′-atgggtaaggargacaagac-3′,;
and
EF2T:
SEQ ID NO: 14
5′-ggargtaccagtsatcatgtt-3′,.

The sequence obtained by sequencing is shown in SEQ ID NO: 15:

5′-ttttttttagtggatatgaggggcaagactcacatcaacgtggtcgttatcgtaagtcgcctgcctccatttcgaactttgtagaagctgttgtact
gacttgcttgtcgtaggggtatgtttcggagcctacactcttcgccgtcccgagtttgtgataactgactggtcctcacagccacgtcgactcc
ggcaagtctaccaccactggtcacttgatctaccagtgcggtggtatcgacaagcgtaccattgagaagttcgagaaggtaagccaaacca
ctccgattaatgatctgctattgtttggcgatgaacattattgggtttcccgctgcctgtcggccattacccctcactgtggcacgaaaattttcgc
ggggccttatcttggactttggtggggcatcataccccgccagctgtcgagggtgtctctgtgtgtctctggctgttgaaaccacaatattgtc
gttgctttcagagggaaaaaacatgaaactaatttggatcgctgtataggaagccgctgaactcggcaagggttccttcaagtacgcatgggt
tcttgacaagctcaaggccgagcgtgagcgtggtatcaccatcgacattgccctctggaagttcgagactcccaagtactatgtcaccgtcat
tggtatgtcgacttgcgcaaactgaccgcagacttttctcctaaattgaatgctaatgcccctcccacagacgctcccggtcaccgtgactttat
caagaac-3′.

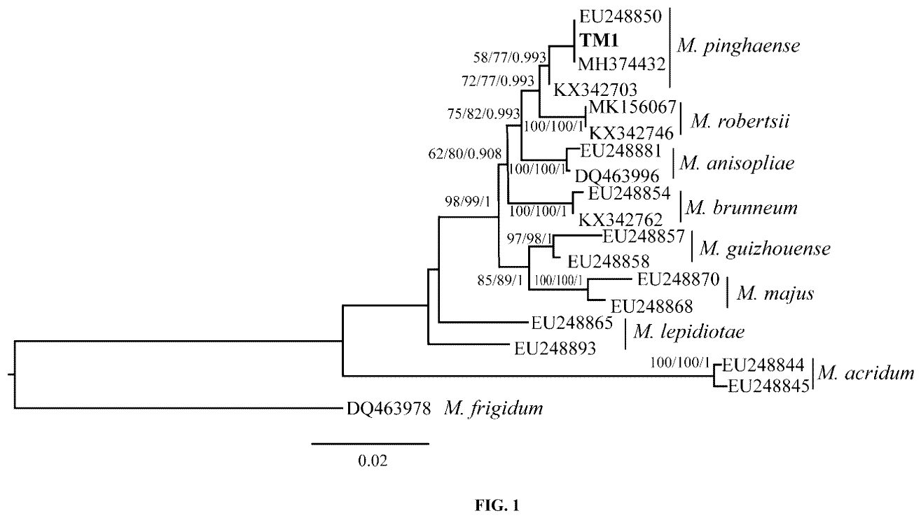

This sequence combined with 5TEF reference sequences of 9 taxonomically validated representative species of the genus Metarhizium were subjected to phylogenetic analysis. The analysis results showed that the strain (TM1) was M. pingshaense (Mp), as shown in FIG. 1. The figure shows the phylogenetic tree constructed by the maximum likelihood method, with the GenBank gene accession number of each sequence displayed alongside the branches. The numbers on the nodes represent the support values of the maximum likelihood method (left), the neighbor-joining method (middle), and the Bayesian inference method (right). A hyphen (-) indicates that there was no support at the node in the corresponding method; the scale represents the estimated number of nucleotide substitutions at each site.

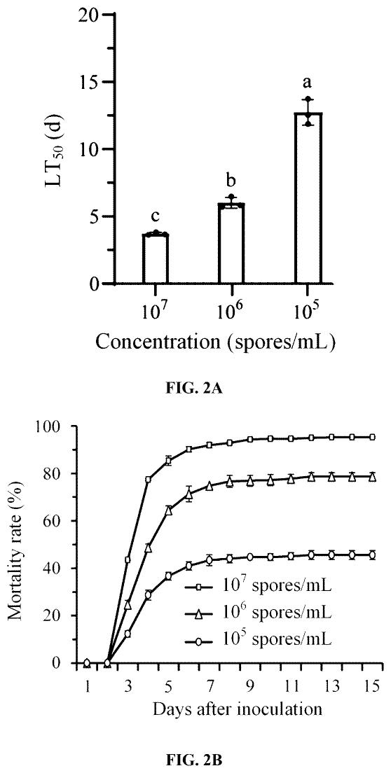

The spores of the M. pingshaense (Mp) cultured on PDA medium for 14 d were suspended and vortexed in 0.01% Triton X-100 and then passed through a glass wool filter to obtain a spore suspension. The spore suspension was subjected to spore concentration determination using a hemocytometer, and then diluted to the required concentrations (105, 106, 10⁷ spores/mL) for inoculation of S. invicta. The inoculation was done by immersion, that is, the workers of S. invicta were picked up and placed in a 50-mL centrifuge tube containing 15 mL of the spore suspension. The tubes were gently shaken for 15 s to immerse all the workers in the spore suspension. The ants were then quickly transferred to absorbent paper to absorb the moisture on the surface of ant bodies. 10 workers were transferred into insect-rearing boxes as a group and then placed in an incubator at 25° C. with a light/dark cycle of 12 h: 12 h. Honey water was placed in the insect-rearing boxes as food and fresh honey water was replaced every 2 d. The death of ants was observed every day, the cadavers were removed in time and kept in a moisturizing culture for 3 d to 5 d to observe whether Metarhizium induced cadavers were formed, and individuals becoming cadavers were counted as mortality. The virulence test was repeated 3 times, with 100 ants tested each time, while 0.01% Triton X-100 was used as a blank control. The statistical results of median lethal time and mortality rate are shown in FIGS. 2A-2B.

The results showed that the screened M. pingshaense strain was a strain with high virulence to S. invicta; when the spore concentration was 10⁷ spores/mL, the LT₅₀ of this strain was only 3.4 d, and the mortality rate of S. invicta reached 95% after 14 d of inoculation. The strain demonstrated superior insecticidal efficacy compared to the reported fungal strains retrieved [1]-[4]. The strain was deposited in the CGMCC, with the deposit number CGMCC NO: 41511.
[1] Metarhizium brunneum, Metarhizium anisopliae: Jin X X, Streett D, Huang Y B, Ugine T. Development of a novel bioassay system to assess the effectiveness of entomopathogenic fungi against imported fire ants. Biocontrol Science and Technology, 2012, 22(2): 233-241.
[2] Isaria fumosorosea, Beauveria bassiana, and Metarhizium anisopliae: Qiu H L, Lu L H, Zhang C Y, He Y R. Pathogenicity of individual isolates of entomopathogenic fungi affects feeding preference of red imported fire ants S. invicta. Biocontrol Science and Technology, 2014, 24(11): 1286-1296.
[3] Beauveria bassiana: Li J, Guo Q, Lin M F, Jiang L, Ye J W, Chen D S, Li Z G, Dai J Q, Han S C. Evaluation of a new entomopathogenic strain of Beauveria bassiana and a new field delivery method against S. invicta. PLOS One, 2016, 11(6): e0158325.
[4] 11 strains of Beauveria bassiana and 9 strains of Metarhizium anisopliae: Wu Z P, Tong Y H. Pathogenicity of Beauveria bassiana and Metarhizium anisopliae to S. invicta workers. Journal of Forestry and Environment, 2020, 40(01): 99-105.

### Example 2 Attraction of Longifolene to S. invicta

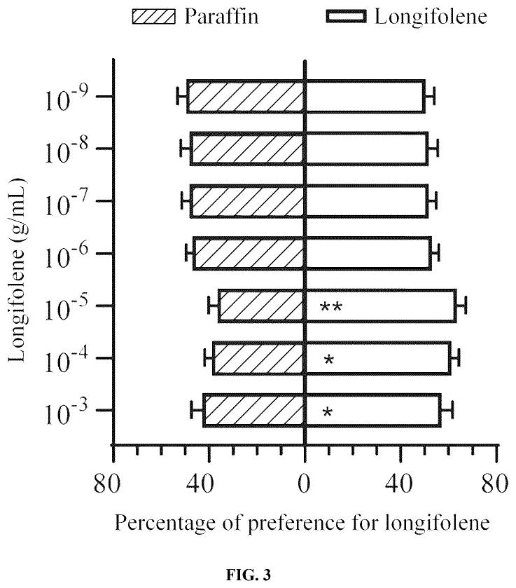

The attraction of longifolene to workers of S. invicta was tested using a two-way selection method. Two circular filter papers, each with a diameter of 6 mm, were placed on opposite sides of a culture dish with a diameter of 15 cm. The two circular filter papers contained 10 μL of paraffin oil and 10 μL of paraffin oil solution containing longifolene, respectively. Longifolene-containing circular filter papers with different concentrations (10-3 g/mL, 10-4 g/mL, 10-5 g/mL, 10-6 g/mL, 10-7 g/mL, 10-8 g/mL, 10-9 g/mL) were set up. 20 healthy and active workers of S. invicta were placed in the middle of the culture dish, and the choice of workers was recorded after 10 min. The experiment was repeated 15 times, and the results are shown in FIG. 3. The results showed that longifolene had an attractive effect on workers of S. invicta, with significant effects observed at concentrations of 10-3 g/mL, 10-4 g/mL, and 10-5 g/mL.

### Example 3 Construction of Plasmid pPK2-Mmd-GFP with Methylmercury Demethylase Gene Mmd of M. pingshaense as Selection Marker Gene

1) The original plasmid vector used for construction was pPK2-Bar-GFP (published in the literature [Zhang et al., Horizontal gene transfer allowed the emergence of broad host range entomopathogens. Proc. Natl. Acad. Sci. U.S.A. 2019, 116, 7982-7989]). The herbicide-resistant Bar gene expression cassette was cut out by restriction endonucleases EcoR I and EcoR V, and the large fragment DNA (pPK2-GFP) produced by enzyme cutting was recovered.

2) With a genome of M. pingshaense (denoted as Mp) screened in Example 1 as a template, PCR amplification was conducted using primers Gpd-F and Gpd-R to obtain the promoter of the 3-phosphoglyceraldehyde dehydrogenase gene Gipd, and then PCR amplification was conducted using primers Mmd-F and Mmd-R to obtain a DNA fragment containing the open reading frame (ORF) and terminator region of the methylmercury demethylase gene Mmd. The primer sequences are as follows:

Gpd-F:
(SEQ ID NO: 1)
5′-ggagatctgatatcactagtattaaacatggcacact-3′;
Gpd-R:
(SEQ ID NO: 2)
5′-atggggctctgttgactcattttgcgtgtgtgtatatgga-3′;
Mmd-F:
(SEQ ID NO: 3)
5′-atgagtcaacagagccccat-3′;
and
Mmd-R:
(SEQ ID NO: 4)
5′-gggaattcattcagaacggagaagt-3′.

The system and procedure for amplifying Gpd and Mmd fragments were the same and were as follows.

The total reaction system was 50 μL, including 4 μL of template genome (concentration 100 ng/μL), 25 μL of 2×Phanta Max Buffer, 1 μL of dNTP Mix (10 mM each), 2 μL each of upstream and downstream primers (10 μM), 1 μL of Phanta Max Super-Fidelity DNA Polymerase, and a balance of double-distilled water (ddH₂O) to make up to 50 μL.

The reaction procedure included initial denaturation at 95° C. for 3 min; denaturation at 95° C. for 30 s, annealing at 56° C. for 15 s, extension at 72° C. for 1 min and 30 s, 32 cycles; and extension at 72° C. for another 5 min.

3) Using the two PCR reaction products obtained in step 2) (the two DNA fragments had a 20 bp overlapping region) as templates, and Gpd-F and Mmd-R as primers, a long DNA fragment (Gpd-Mmd) containing the Gpd promoter, Mmd gene ORF, and terminator was amplified. The amplification system and program were as follows.

The total reaction system was 50 μL, including 4 μL each of the Gpd and Mmd fragments obtained in step 2 (concentration of each 100 ng/μL) as template DNA, 25 L of 2xPhanta Max Buffer, 1 μL of dNTP Mix (10 mM each), 2 μL each of upstream and downstream primers (10 μM), 1 μL of Phanta Max Super-Fidelity DNA Polymerase, and a balance of ddH₂O to make up to 50 μL.

The reaction procedure included initial denaturation at 95° C. for 3 min; denaturation at 95° C. for 30 s, annealing at 56° C. for 15 s, extension at 72° C. for 2 min and 40 s, 32 cycles; and extension at 72° C. for another 5 min.

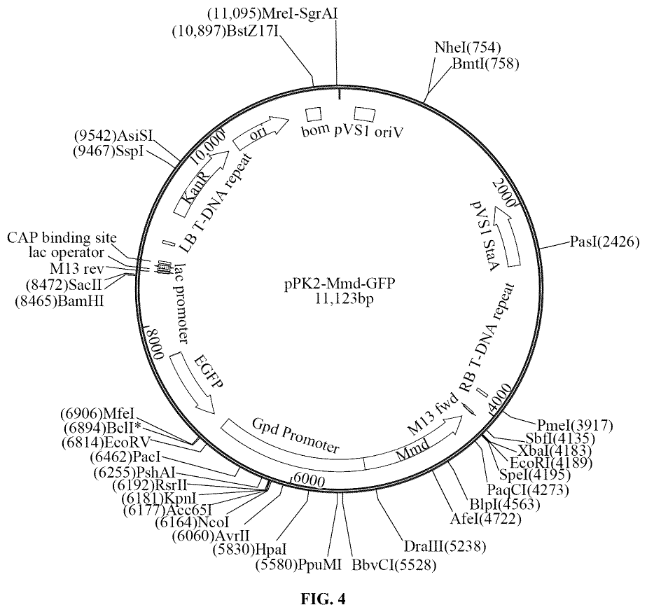

4) The DNA fragment Gpd-Mmd obtained in step 3) was digested with EcoR V and EcoR I, and ligated with the fragment pPK2-GFP obtained in step 1) by digestion with EcoR V and EcoR I to obtain a recombinant plasmid pPK2-Mmd-GFP (FIG. 4), where the selection marker gene was the methylmercury demethylase gene Mmd (SEQ ID NO: 5) of M. pingshaense, and its expression was driven by the promoter of the 3-phosphoglyceraldehyde dehydrogenase gene Gpd (SEQ ID NO: 6) of M. pingshaense. The specific sequences were as follows.

The Mmd gene had the nucleotide sequence shown in SEQ ID NO: 5:

5′-atgagtcaacagagccccatcaagattcaactgctgaccgttccagactgcccgttggtagccaaggtccgtgacacactcaacaactgctt
ggcaaaaacacgctcaggcgcaacggtcgaggagcttgtgggagagtatcattctccaacgcttctcatcaacggcttcgacgtcaccggt
aagcccgtctcagcacaaggacaacaaagctgccgtctcgatcttcccaatgaggagcagatacttgctgctcttcggggcctgcccgttct
aagttgcgaggatgaaacagaggcagcggtgggaaagtctgcttttcacattctgctacgtaccgcagggcgtgtcgcattggaacaggtc
agccaagagactggtagaaatacggacgacatcaggaccggtatagaggcgcttcggcgcaggggccatgtcaagatagataaacaag
gcttcatcattggggttgctggattgagttgcattcccacagagcatcaactttccatcgaggggaagagactctgggcttggtgtgcctttga
cgtgattggcatttttggtgctcttgaggcgtctggattcgcgacatcggcagacccggctactaacgagcgcttggtcgtcaactttgtcaag
ggtgttcctgatgagacggggcttggggttttcatggcagacatgccaccggggggctctgtctgcgaggactggtgttggagagtgcgat
tttttcagtccgagtcggcggctgaggcttgggcccgggcgaacggtgtcacgggttctcttatatctgtggcaaacctgatggtatcggccc
gcgaagcatggagtaggtacgggttgtcttgaggatagactggcggaattggtatgacctgccatctgagagcgggaagctccatgcaagt
catagttctctcagctctgtaaagatgacttcttgttcgtgttttctatggtgtgttcgtgatagcagagtggcggttcagaagaaataatgattcta
gctactaggtggacgtttcttaattaaaccggtcgacatgtccaagtacacagttgctctctccgaaatgtccgcattcacttctccgttctgaat-
3′;

and

the promoter of the gpd gene had the nucleotide sequence of SEQ ID NO: 6:

5′-acatggcacactggtggagacgatttcatggatggcaagactaagcattgggcccagacacgggggttgacacacgggcaggttgcccc
ggtccaggtggaagtttacgattgaaaacttccgaagagcttgtattccctgtgggcaaagttcgggacgacgagaactgttcctggtgttcc
cgacgactgtccccgggggacgcggctgcaaaggccagagccgcccgcagccgtgaagctccgggaaagatgggcaaaacgggc
gaatggatggccaatacagatgaaccatttcccgccatgcatccgagccgcccacgcgaccagaggggccccggaattaattagattgag
gtcatcgtgaaggcattccgctgctgagagtgcgacagagctggcgtcggtacattttggacatgtctgggccgtctgatttttttttccgtctc
ctctcacacctgactgcgtgagtcagcagcaagtcgtcaccgcgaccggagacacgttttgagtgatttatcatggctattactcgcggtcttg
ctgccagcaggcatatctacaacacgataaggccagcagggcctaccacgggggtacatggacgccaggctgcgacaaccatggaaca
gcgggtacccctcatgtccatgttttttttctctctctcaaggtggtgctcatgtgctcgtcggtgtggttgagagagcgtttctattaacgaattgt
gtccccgccggatccgtaaaaaagacgcatcggctgggtggagcaaggcgaggcccgtcaaagcatcatcaatggctgagctcattcgat
tcctcatcaaattacatggcgcagggaagaatcgctggcgaattttgcctttgcggtgcatgcaagtggcggctggctgggatattggatgg
ccacttgcacgaccacggcgaggatggacatgaagcccgcggcgtctggcatggcaaggcaattgaaatatcgtctcgttgaatagcgtg
aatagtagtaccgctcgtgtatcagaacccggctccgtctcacggtgcaagtgtgaatgatgcatgcacccgcttggctaggtttgcgtgcac
cagttgttggccgctgacgtctgctggtctcaacagtcccgacgccgtggccagccaagacgcagccagttgcgcgcccagcaacacac
cagacaccccaagcaccagacccaagctcccaagctccagggacatggactgaccactattttgtcccgccttccctccgtcccctccgac
tccatcttcttccctcttcctcttcctttttcttcttctcttcccgctctccatacaacttcaccattcttcccaccaggcactttgtccggtatgtatccc
gtcctgcattggctgagcagagcttgaccacagctcgacaccagctccgtctctagcttcccagactcccgtgctgacgtccaagctccagc
tctctccatatacacacacgcaaa-3′.

### Example 4 Construction of Engineered Strain Mmd-Tps of M. pingshaense that Produces Longifolene and Kills S. invicta

The TPS coding sequence obtained by digestion with restriction endonucleases BamH I and EcoR V in Chinese patent CN116904329A was ligated to the vector pPK2-bar-Ptef (Zhang X, Meng Y M, Huang Y Z, Zhang D, Fang W G. A novel cascade allows Metarhizium robertsii to distinguish cuticle and hemocoel microenvironments during infection of insects. PLOS Biol. 2021 Aug. 4; 19(8): e3001360), and then digested with the same enzymes to obtain the Tps expression vector pPK2-bar-Tps-Ptef. Using pPK2-bar-Tps-Ptef as a template and Tef-EcoRI-F (SEQ ID NO: 7) and Trpc-XbaII-R (SEQ ID NO: 8) as primers, PCR amplification was conducted to obtain the PTef-Tps-TTrpc fragment (containing constitutive promoter pTef, longifolene synthase ORF, and terminator Ter). Primer sequences were as follows.

Tef-EcoRI-F:
(SEQ ID NO: 7)
5′-ccgaattctagcaacagtcagctagacg-3′;
and
Trpc-XbaI-R:
(SEQ ID NO: 8)
5′-gctctagagcattgcagatgagctgtat-3′.

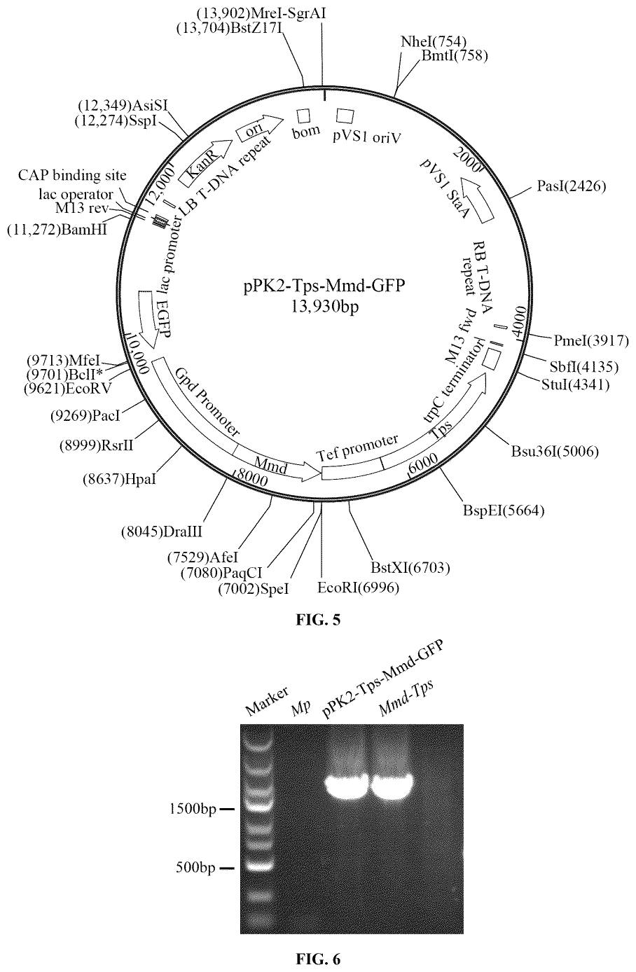

The PTef-Tps-TTrpc fragment was digested with restriction endonucleases EcoR I and Xba I, and ligated to the vector pPK2-Mmd-GFP (constructed in Example 3) double-digested with the same enzymes to obtain an expression vector pPK2-Tps-Mmd-GFP of the Tps gene (FIG. 5).

The pPK2-Tps-Mmd-GFP plasmid was transferred into the Agrobacterium tumefaciens AGL1 strain, and then the genetic transformation of M. pingshaense mediated by Agrobacterium tumefaciens was conducted to obtain the engineered strain Mmd-Tps. The transformation was conducted according to the reference (Xu C, Zhang X, Qian Y, Chen X, Liu R, Zeng G, Zhao H, and Fang W. A high-throughput gene disruption methodology for the entomopathogenic fungus Metarhizium robertsii. PLOS One. 2014, 9(9):e107657), including the followings.

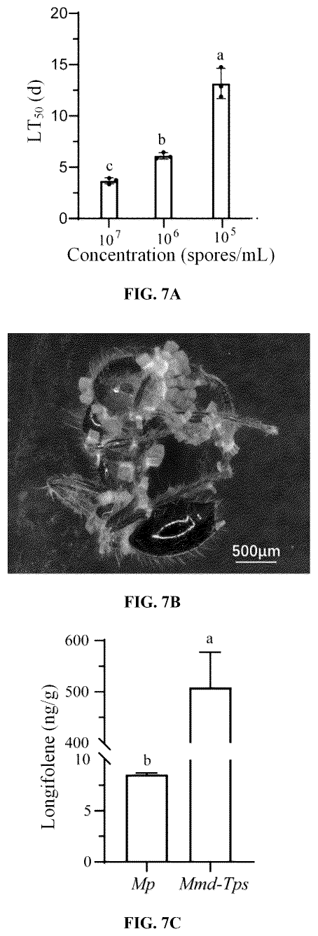

Equal volumes of Agrobacterium tumefaciens culture solution and M. pingshaense spore suspension were gently vortexed to mix, and evenly spread on an induction solid medium (referring to literature) covered with black sterile filter paper (Macherey-Nagel, Germany) and containing 200 μmol acetosyringone (AS), and cultured upright in a 26° C. incubator for about 2 d until white fuzzy hyphae grew on the black filter paper. The black filter paper was transferred to M-100 solid medium containing 400 μg/mL cefotaxime and 10 μg/mL methylmercury and covered with the same medium. After 3 d to 5 d, single colonies that might be transformants were picked and plated on M-100 solid medium containing 10 μg/mL methylmercury, and secondary screening was conducted in combination with the green fluorescent protein (GFP). Transformants with green fluorescence were screened by observation, and a small amount of hyphae was selected as a template for fungal PCR verification to confirm the insertion of the Tps gene into the genome of M. pingshaense to successfully construct the engineered strain Mmd-Tps. The primers used were Tps-F (SEQ ID NO: 9) and Tps-R (SEQ ID NO: 10), and the PCR verification results are shown in FIG. 6. Primer sequences were as follows:

Tps-F:
(SEQ ID NO: 9)
5′-atggcccagatctccatc-3′;
and
Tps-R:
(SEQ ID NO: 10)
5′-ttagatggggacctggtcg-3′.

### Example 5 Analysis of Virulence of Engineered Strain Mmd-Tps to Workers of S. Invicta, Amount of Longifolene Volatiles from Ant Cadavers, and Attraction to Workers

### 1. Methods

Virulence determination method: the determination of the virulence of Mmd-Tps to workers of S. invicta was consistent with the M. pingshaense strain screened in Example 1.

Detection method of the volatile amount of longifolene: when the workers of S. invicta inoculated with M. pingshaense died, ant carcasses were disinfected with sodium hypochlorite solution (0.4%) and cultured in a moisturizing manner for 7 d to form cadavers covered with mycelium. 0.5 g of ant cadavers were placed in a sample injection bottle and inserted into an extraction head (50/30 μm DVB/CAR/PDMS) for adsorption for 40 min, during which time the sample injection bottle was placed in a 40° C. water bath. The volatile substances adsorbed on the extraction head were analyzed by SPME-GC-MS. Manual injection was adopted at the injection port, the injection port was at 250° C., the desorption was conducted for 3 min, and the analysis was done using a DB-5 MS (30 m×0.25 mm, 0.25 μm) chromatographic column. The GC-MS program was as follows: the initial temperature was 40° C. and maintained for 2 min; the temperature was increased to 180° C. at 5° C./min, and then increased to 270° C. at 10° C./min and maintained for 10 min. According to the total ion current (TIC) diagram, the mass spectrum data of the chromatographic peak was compared with the mass spectrum library NIST05 to preliminarily predict the chromatographic peak compounds. According to the CAS number of the compound provided by the mass spectrometry library, a chemically synthesized standard was purchased, and the standard and a C₇-C₄₀ normal alkane mixed standard were analyzed by GC-MS using the above procedure. The linear retention index (LRI) was calculated according to the LRI calculation formula (Formula I):

$${RI}_{x} = 100 \times z + 100 \times ( {RT}_{x} - {RT}_{z} ) / ( {RT}_{z + 1} - {RT}_{z} ) ; \qquad Formula I$$

RIₓ represents the linear retention index of the target component x, z represents the number of carbon atoms contained in the n-alkane before the target component x eluted, RTₓ represented the retention time of the target component x, RT_{z} represents the retention time of the z-th carbon n-alkane, and RT_{z+1} represents the retention time of the z+1-th carbon n-alkane.

The retention index confirmed that the compound was the same as the standard. Finally, the volatile amounts of longifolene from the ant cadavers infected by the wild-type strain (Mp) and Mmd-Tps were calculated based on the confirmed longifolene peak areas.

Method for determining the attraction to workers: the workers were frozen to death in a −80° C. freezer for later use, while ant cadavers formed by infection with the wild-type strain Mp and the engineered strain Mmd-Tps were used for detection using the two-way selection method. 0.1 g of each type of frozen and infected cadavers were placed on both sides of a culture dish with a diameter of 15 cm, and then 20 healthy and workers of S. invicta were placed in the middle of the culture dish. After 10 min, the number of healthy workers that chose each side was recorded, and the response index (RI) was calculated, RI=(the number of workers that chose cadavers—the number of workers that chose frozen ant carcasses)/the total number of workers. In order to examine the preference of healthy workers for cadavers infected with the wild-type strain and the engineered strain Mmd-Tps, the two types of cadavers (0.1 g each) were placed on both sides of a culture dish and tested using a two-way selection method, and the percentage of preference was calculated.

### 2. Results

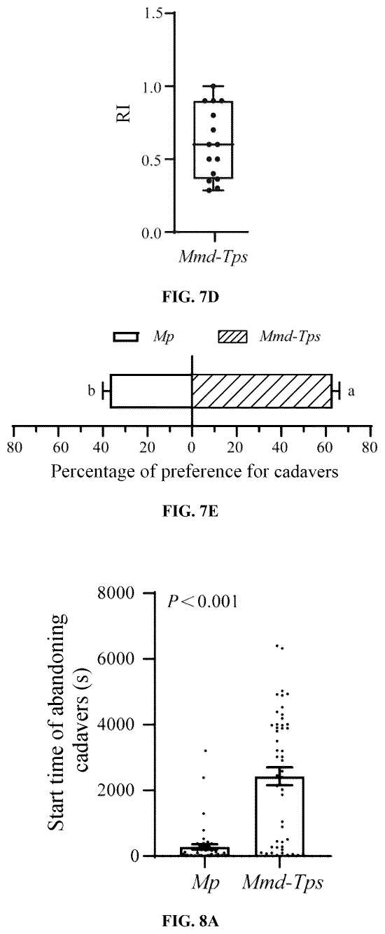

The LT₅₀ of the engineered strain Mmd-Tps was not significantly different from that of the wild-type M. pingshaense strain (P>0.05) (FIG. 7A). Workers infected by Mp and Mmd-Tps showed the same typical fungal lethal symptoms, namely, dense hyphae grew on the surface of the ant carcasses after 3 d of moisturizing culture, and then began to produce spores (FIG. 7B), indicating that the expression of the Tps gene did not affect the virulence of M. pingshaense strain to S. invicta.

Both the engineered strain Mmd-Tps and the wild-type strain could result in longifolene volatilization, but the longifolene volatilization induced by the Mmd-Tps strain was 59 times higher than that of Mp, reaching 509 ng/g cadaver (FIG. 7C).

The Mmd-Tps induced zombie ant had a significant attracting effect on workers of S. invicta, with a response index of 0.61 (FIG. 7D). The preference experiment showed that healthy workers preferred Mmd-Tps induced cadavers to Mp induced cadavers (63% vs. 37%) (FIG. 7E).

### Example 6 Effect of Engineered Strain Mmd-Tps on Hygienic Behavior of Workers of S. invicta

### 1. Methods

In order to analyze the abandonment behavior of workers towards ant cadavers formed by Mp and Mmd-Tps infection in S. invicta workers, and the probability of being inoculated with spores from ant cadavers during this process, artificial ant colonies were established in insect-rearing boxes (30×30×15 cm). Each worker colony consisted of 500 workers, with 20 larvae, and 1 reproductive ant. 50 ant cadavers were placed in the box of each artificial ant colony, and the time when the workers began to abandon cadavers was recorded, where the ant cadavers were obtained by moisturizing and culturing for 7 d after infection with Mp and Mmd-Tps suspensions with a concentration of 10⁷ spores/mL. 24 h later, 50 workers were randomly selected, ground and coated on the screening medium of Metarhizium, and cultured at 26° C. for 5 d. The colony forming units (CFUs) were calculated, and the number of spores attached to the body surface of each worker was expressed according to the number of spores. This experiment was repeated 5 times.

### 2. Results

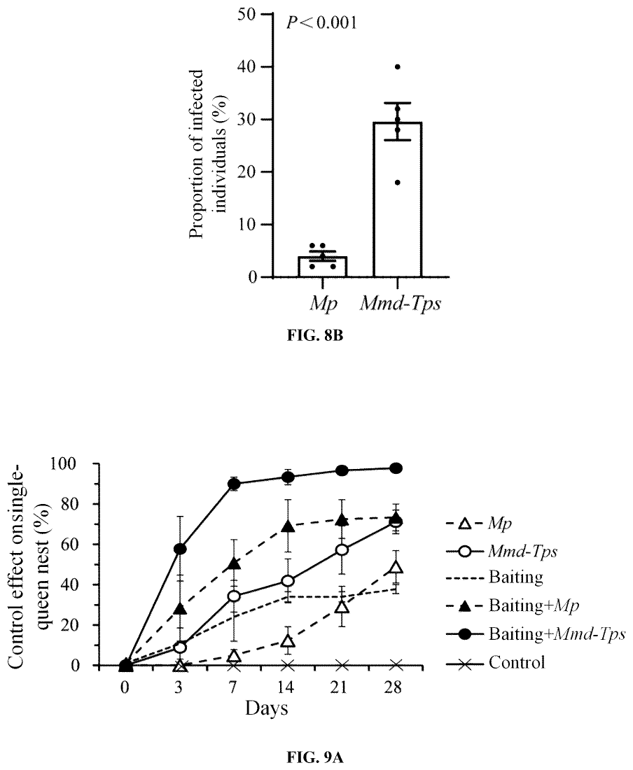

Healthy workers began to abandon Mp induced cadavers in less than 5 min (average 282 s), while the starting time of abandoning Mmd-Tps induced cadavers was delayed 8.6 times (average 2,429 s) (p<0.001) (FIG. 8A). After 24 h, only 4% of the workers carrying Mp induced cadavers were inoculated with spores, while the proportion of workers carrying Mmd-Tps induced cadavers that were inoculated increased by 7.5 times (30%). This was because the Mmd-Tps had a stronger attraction to the workers, which prolonged the contact time between the workers and the ant cadavers (FIG. 8B).

### Example 7 Preparation of Wettable Powder of M. pingshaense

The Metarhizium spores are hydrophobic on surface and difficult to be directly suspended in water. For ease of use, the present disclosure provided wettable powder of M. pingshaense, including the following components in percentage by mass: 35% M. pingshaense spore powder, 57% talc, 3% sodium dodecyl sulfate (SDS), and 5% sodium salt of polynaphthalene sulphonic acid (dispersant NNO).

The wettable powder was dissolved in water to prepare a spore suspension with a final concentration of 1×10⁷ spores/mL during use, and then poured into an ant nest to infect S. invicta.

### Example 8 Combination of Engineered Strain Mmd-Tps and Hydramethylnon Bait to Control S. invicta

### 1. Methods

The experiment for controlling S. invicta nests (single-queen type and multiple-queen type) was set up with the following 6 treatments.
(1) 1×10⁷ spores/mL of wild-type strain Mp spore suspension was injected into each nest, 2 L per nest, designated as Mp.
(2) 1×10⁷ spores/mL of engineered strain Mmd-Tps spore suspension (the spore suspension prepared in Example 7) was injected into each nest, 2 L per nest, designated as Mmd-Tps.
(3) Treatment with 0.73% hydramethylnon bait was conducted (20 g per nest, evenly sprinkled on the ant nest, the same below), designated as baiting.
(4) After treatment with 0.73% hydramethylnon bait, 2 L of Mp spore suspension with a concentration of 1×10⁷ spores/mL was injected into each nest 3 d later, designated as baiting+Mp.
(5) After treatment with 0.73% hydramethylnon bait, 2 L of Mmd-Tps spore suspension with a concentration of 1×10⁷ spores/mL was injected into each nest 3 d later, designated as baiting+Mmd-Tps.
(6) 0.01% Triton X-100 was poured, 2 L per nest. This treatment was a blank control and designated as the control.

The wild-type strain Mp spore suspension was similar to the spore suspension prepared in Example 7, except that the engineered strain Mmd-Tps was replaced by the wild-type strain Mp.

The experiment was conducted in an insect-proof mesh room, with 15 ant nests in each treatment and repeated 3 times. To prevent nest relocation caused by human interference, the spore suspension should be slowly poured into the mound. The mortality of ant nests was observed before treatment and 3, 7, 14, 21 and 28 d after treatment. The surface of the nest was tapped for 1 min, and any nest with less than 3 workers emerged during this period was considered inactive. Nest relocation and colony fission in the treated ant nests were observed, and the ant nest control effect was calculated based on the mortality, nest relocation, and colony fission in the ant nests.

$$Ant nest control effect ( \% ) = ( 1 - number of active nests after treatment / number of inactive nests before treatment ) \times 100.$$

### 2. Results

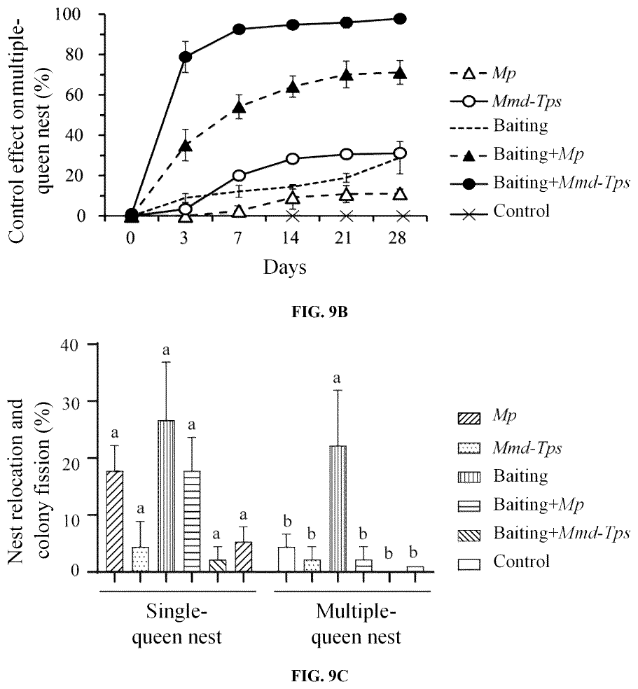

The test results (FIG. 9A to FIG. 9C) showed that there was no significant difference in control effects between using only hydramethylnon bait and using only the wild-type strain Mp. After 28 d of both treatments, the control effect on single-queen nest was about 40%, with colony fission and nest relocation accounting for 20% to 30%. For multiple-queen nest, the control effect was less than 30%. Specifically, after treatment with hydramethylnon bait, the control effect was 28.9%, with 20% of the ant nests being relocated. After treatment with wild-type strain Mp, the control effect was only 11.1%. The control effect of using Mmd-Tps alone was significantly higher than that of using Mp alone. After 2 weeks of treatment, the control effect of Mp against single-queen nest was 12.3%, while the control effect of Mmd-Tps was 41.8%. After 28 d, the control effect of Mmd-Tps reached 71%. Mmd-Tps also had a high control effect against multiple-queen nests, reaching 31% after 28 d. Both strains induced nest relocation or colony fission behaviors upon inoculation, but there was no significant difference between the two groups. The combined use of hydramethylnon bait and M. pingshaense significantly improved the control effect. After 1 week of treatment, the combined use of Mp and bait had a control effect on single-queen nests of 51% (54% for multiple-queen nest); after 28 d of treatment, the control effect on single-queen nests was 73% (71% for multiple-queen nests). The combination of engineered strain Mmd-Tps and bait had a higher control effect, and nest relocation or colony fission was rarely seen; after 3 d of treatment, the control effect on single-queen nest reached 58%, and the control effect on multiple-queen nests was as high as 79%. After 7 d, the control effect on multiple-queen nests was 93% (90% for single-queen nests); after 28 d, both social types of ant nests showed a control effect of 98%, with 4 out of 6 tests achieving a 100% control effect.

Although the above examples have described the present disclosure in detail, it is only a part of, not all of, the embodiments of the present disclosure. Other embodiments may also be obtained by persons based on the example without creative efforts, and all of these embodiments shall fall within the protection scope of the present disclosure.

What is claimed is:

1. A method for constructing an engineered strain for controlling Solenopsis invicta (S. invicta), comprising using a Metarhizium pingshaense (M. pingshaense) strain as a starting strain, wherein the M. pingshaense strain has a deposit number of CGMCC NO. 41511.

2. An engineered strain for controlling S. invicta, comprising a starting strain and a longifolene synthesis gene Tps introduced into the starting strain; wherein the starting strain comprises an M. pingshaense strain having a deposit number of CGMCC NO. 41511; and the longifolene synthesis gene Tps encodes a protein having the amino acid sequence of SEQ ID NO: 11.

3. The engineered strain according to claim 2, wherein the engineered strain further comprises a selection marker gene introduced into the starting strain; the selection marker gene is a methylmercury demethylase gene Mmd of the starting strain, and the methylmercury demethylase gene Mmd has the nucleotide sequence of SEQ ID NO: 5.

4. The engineered strain according to claim 3, wherein the methylmercury demethylase gene Mmd is driven by a promoter of a 3-phosphoglyceraldehyde dehydrogenase gene Gpd of the starting strain, and the promoter has the nucleotide sequence of SEQ ID NO: 6.

5. A microbial inoculant for controlling S. invicta, comprising spore powder, a solid phase carrier, an emulsifier, and a dispersant; wherein the spore powder comprises spore powder of an M. pingshaense strain having a deposit number of CGMCC NO. 41511 and/or spore powder of the engineered strain according to claim 2.

6. The microbial inoculant according to claim 5, wherein the engineered strain further comprises a selection marker gene introduced into the starting strain; the selection marker gene is a methylmercury demethylase gene Mmd of the starting strain, and the methylmercury demethylase gene Mmd has the nucleotide sequence of SEQ ID NO: 5.

7. The microbial inoculant according to claim 6, wherein the methylmercury demethylase gene Mmd is driven by a promoter of a 3-phosphoglyceraldehyde dehydrogenase gene Gpd of the starting strain, and the promoter has the nucleotide sequence of SEQ ID NO: 6.

8. The microbial inoculant according to claim 6, wherein the solid phase carrier comprises talc; the emulsifier comprises sodium dodecyl sulfate (SDS); and the dispersant comprises sodium salt of polynaphthalene sulphonic acid.

9. A method for controlling S. invicta, comprising the following steps: applying a bait for luring and killing the S. invicta to a surface of an S. invicta nest, and then adding a spore suspension to the S. invicta nest after the bait is carried by an ant colony; wherein a spore in the spore suspension is one or more selected from the group consisting of a spore of an M. pingshaense strain having a deposit number of CGMCC NO. 41511, a spore of the engineered strain according to claim 2, and spore powder in a microbial inoculant.

10. The method according to claim 9, wherein the bait for luring and killing the S. invicta comprises hydramethylnon.

11. The method according to claim 9, wherein the engineered strain further comprises a selection marker gene introduced into the starting strain; the selection marker gene is a methylmercury demethylase gene Mmd of the starting strain, and the methylmercury demethylase gene Mmd has the nucleotide sequence of SEQ ID NO: 5.

12. The method according to claim 11, wherein the methylmercury demethylase gene Mmd is driven by a promoter of a 3-phosphoglyceraldehyde dehydrogenase gene Gpd of the starting strain, and the promoter has the nucleotide sequence of SEQ ID NO: 6.

13. The method according to claim 9, wherein the microbial inoculant comprises spore powder, a solid phase carrier, an emulsifier, and a dispersant; wherein the spore powder comprises spore powder of an M. pingshaense strain having a deposit number of CGMCC NO. 41511 and/or spore powder of the engineered strain.

14. The method according to claim 13, wherein the solid phase carrier comprises talc; the emulsifier comprises sodium dodecyl sulfate (SDS); and the dispersant comprises sodium salt of polynaphthalene sulphonic acid.
